(19) United States
(12) Patent Application Publication
(10) Pub. No.: US 2017/0325996 A1
(43) Pub. Date: Nov. 16, 2017

(54) Nasolacrimal Compression Device

(71) Applicant: Stephen C. Chen, Weston, MA (US)

(72) Inventor: Stephen C. Chen, Weston, MA (US)

(21) Appl. No.: 15/152,546

(22) Filed: May 11, 2016

(51) Int. Cl. A61F 9/00; A61B 17/00

(52) U.S. Cl. CPC A61F 9/0008; A61B 2017/00004; A61F 2009/0043

(57) ABSTRACT

A device is used to temporarily compress the nasolacrimal ducts to reduce outflow of eyedrop during and after eyedrop instillation. The device is to be hand bended to snuggly fit any adult head sizes with any facial features so that the nose pads may apply firm pressure over the nasal aspect of the orbital rim where nasolacrimal ducts lay underneath the skin. The pressure delivered by the device is consistent and is controllable by the user. The device includes failure mechanism to protect against excessive pressure. By keeping eyedrops in the eye, medication absorption is improved and bitter taste due to eydrop outflow is reduced or eliminated. It alleviates the burden of manual nasolacrimal occlusion currently practiced by eye patients and is best for frequent, multiple eyedrops users.

### BACKGROUND OF THE INVENTION

The present invention relates generally to medical device. More particularly, it relates to temporarily occlude the nasolacrimal ducts (also known as nasolacrimal occlusion) in adult patients to reduce outflow through the nasolacrimal ducts. Applying gentle pressure on the skin over the nasolacrimal ducts can effectively reduce tear drainage into the rest of the body system.

Eyedrops are topical ocular medications commonly used for treating eye diseases. The medication is delivered into the eye through absorption. Some eyedrops only need brief time for eye to absorb them while others require much longer time. Ophthalmologists usually advise patients to practice a manual nasolacrimal occlusion procedure by using the index finger to press on the nasolacrimal duct for 5 minutes during eyedrop instillation to keep eyedrops in the eyes long enough to allow full absorption. The benefits of this practice have been recognized for many years. As the nasolacrimal ducts are occluded during eyedrop instillation, the intraocular penetration of topically applied medications is improved for the eye has more time to absorb medications and systemic absorption (which may be toxic) is discouraged for the outflow through the nasolacrimal duct is decreased. However, this procedure puts a big burden on patients and therefore patient compliance is low and the medication efficacy is compromised. To overcome this short coming, several devices had been invented to cope with this problem.

U.S. Pat. Nos. 6,994,684, 6,290,284, 580,317, 5,723,005, 5,334,137, 5,283,063 and 4,959,048 A disclose nasolacrimal occlusion via invasive punctal plugs. The subject of nasolacrimal occlusion via external pressure is disclosed in the following patents:

U.S. Pat. No. 515,872 to Martin et al. discloses a clamp for placing over the nose bridge prior to ocular medication, to seal the nasolacrimal sac to prevent draining of medications away from the eye. The clamp is positioned by a flexible molded nose cover. In one embodiment, the clamp is attached to eyeglasses designed to accept eyedrop applied to the eye.

U.S. Pat. No. 5,832,930 to Martin et al. discloses essentially the same invention but added modified eyeglass frame and elastic fastening band. The limitations are the nose clap with multiple spring selections and elastic fastening band do not offer personalized pressure setting applied on the nasolacrimal ducts. Also, by relying on custom made nose cover to position nasolacrimal ducts is not practical for mass distribution of the device.

U.S. Pat. No. 5,522,837 to Latina discloses a U-shape device, with a bulbous element on the end of each leg that performs the similar function as the nose clamp except it requires hand positioning and pressing. The said device also has a pair of tubes attached to it to channel the eyedrop onto the eyes. The limitation of the U-shape device is that it relies on user to locate the nasolacrimal ducts and it is not adjustable to fit different nose width. Furthermore, it does not offer mobility while using the device.

U.S. Pat. No. 8,147,467 B2 to Stephen C. Chen discloses a device for occluding nasolacrimal ducts but it does not have required flexibility, features, components and methods to support clinical and/or in-home use.

These prior inventions are not practical for clinical and/or in-home use because a pressure based nasolacrimal occlusion device would only work if it had the flexibility to fit user seamlessly in order to apply firm pressure over nasolacrimal ducts.

This invention discloses a novel medical device that FDA haven't had classification for it until Apr. 20, 2016 when FDA granted my De Novo requesting classification of the subject device. Subsequently, the classification was posted on FDA website on May 2, 2016. The classification briefly describes the definition and utility of this type of devices but not specific to the subject device. This invention reveals the details of the actual device.

Therefore, herein disclosed invention overcomes all the limitations of prior arts.

### BRIEF SUMMARY OF THE INVENTION

Since the manual nasolacrimal occlusion procedure suggested by ophthalmologists is too burdensome for patients to follow, this invented device is to alleviate such burden and is best for multiple eyedrop users. This device is to be used to temporarily occlude the nasolacrimal ducts via mechanical pressure in adult patients to reduce outflow through the nasolacrimal ducts. The device should be first fitted by an ophthalmologist to make sure it fits the nasal aspect of patient's orbital rims and to adjust proper pressure on nasolacrimal ducts. An incorrect fitting will let eyedrop leak through nasolacrimal duct. A dye disappearance test, done at doctor's office, may be used to detect if there is any leak. The design concept of this device was developed through years of testing and in-home trials including determination of required stiffness of the device frame, personalized compression pressure, service life and biocompatibility of skin contact components.

A good fitting of an external nasolacrimal duct compression device is very important for successful nasolacrimal occlusion. This invented device is designed to be completely flexible for fitting any sizes and any features of adult human head. This device offers adjustments for nose width, facial width, head size, depth of facial contour, depth of set eye as well as compression pressure. For safety, the device frame is made of specific material which limits occlusion pressure within a safe range.

This invention also discloses methods to verify the device fitting and occlusion effectiveness.

Without proper nasolacrimal occlusion, the eyedrops may outflow through nasolacrimal ducts into the throat causing bitter taste which most patients complained about. However, this bitter taste can be utilized to verify if the device fits the patient correctly. This kind of leak test can be done in home as oppose to the dye disappearance test must be done in doctor's office. Wearing a well fitted device should reduce most of the outflow leak.

This invented device comprising a plastic coated steel wire frame, plastic coated steel wire hooks, non-latex rubber sleeves and hook and loop bands. The steel wire is soft enough to be formed by hands yet strong enough to maintain its shape for normal use. The curved nose pad segments are covered with non-latex rubber sleeves and joined together at the central point. The curved nose pad segments can be bended inwards to increase their curvature to fit deeper facial contour or bended outwards to decrease their curvature to fit flatter facial contour. The distance between two curved nose pad segments can be adjusted to fit either wider or narrower nose. The curved nose pad segments are extended side way to form a pair of frontal segments and then angled backwards to form a pair of temple segments. The frontal segments can be bended upwards to fit narrower face. The frontal segments can also be twisted clockwise to fit deeper set eyes or counterclockwise to fit shallower set eyes. The hook and loop fastening bands are fixed on temple hooks by rings. The temple hook is attached to the temple segment by inserting both temple segment and temple hook into a non-latex rubber sleeve. As the temple hook can slide in and out inside the sleeve, the temple length can be adjusted to fit various sizes of adult head.

Pressure is applied on the nasolacrimal ducts by fastening the hook and loop bands around the back of the head. Excessive tightness will not produce unsafe pressure as the maximum pressure the device can deliver is limited by the yield stress limit of the device frame.

The current invention has many advantages over prior devices that perform similar functions:

1. The disadvantage of nose clamp in one of the prior patents is that the clamp tends to slip away from the nose due to the angular shape of the nose bridge, especially for people with a very shallow or flat nose bridge. This invented device seats on nasolacrimal ducts firmly, disregarding the height of the nose.
2. The devices disclosed in prior patents are not adjustable by hands. This invention offers user full range of hand adjustments for proper fitting.
3. The prior inventions offer either limited or no control over occlusion pressure. This invention offers users full control over occlusion pressure; user can adjust the pressure instantly based on leak test result. And this invented device includes a failure mechanism to protect against excessive pressure.

### BRIEF DESCRIPTION OF THE SEVERAL VIEWS OF THE DRAWING

FIG. 1 is the side view of a person wearing the device.

FIG. 2 is the anatomical site for applying the device.

FIG. 3 is the detailed description of the device.

FIG. 4 is the adjustable components and adjustment methods of the device.

### DETAILED DESCRIPTION OF THE INVENTION

This device frame is made of plastic coated soft carbon steel wire with skin contact portions covered with non-latex rubber sleeves. It is almost identical to a spectacle frame but is fully adjustable. The device frame width, nose bridge frame width, nose pad curvature and temple length can all be adjusted by hands to fit individual users.

Referring now to FIG. 1, the side view of a person wearing the device. The device frame has a central point 1 where curved nose pad segment 2 meet. The curved nose pad segment extends to the frontal segment 3 and the frontal segment extends to the temple segment 4. The hook and loop fastening band 5 go around the back of person's head.

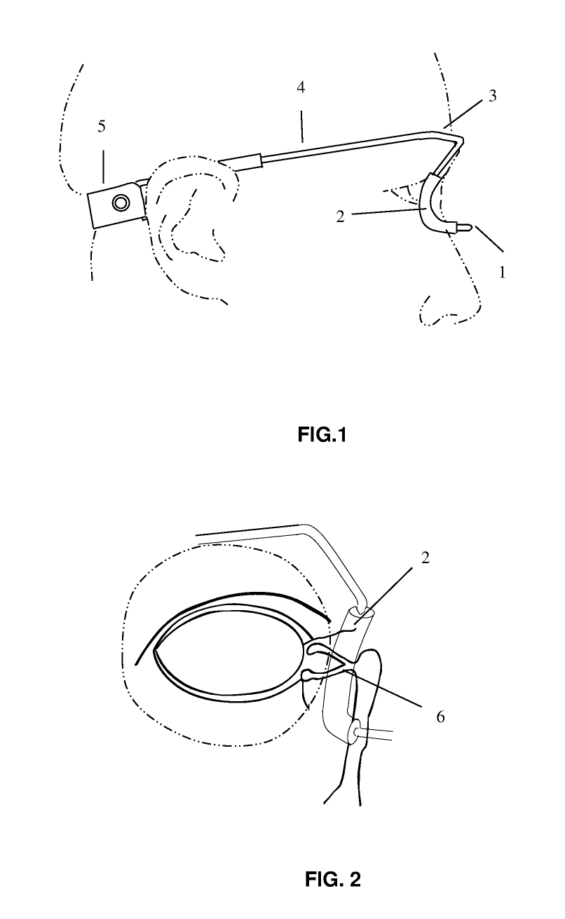

Referring now to FIG. 2, the anatomical site for applying the device. The nose pad 2 sits on top of the nasolacrimal duct 6. The nose pad is shaped concave inward to fit the contour of the face in the nasal aspect of the orbital rim. The graphic illustrates a nose pad sitting across the nasolacrimal duct; exerting gentle pressure on it.

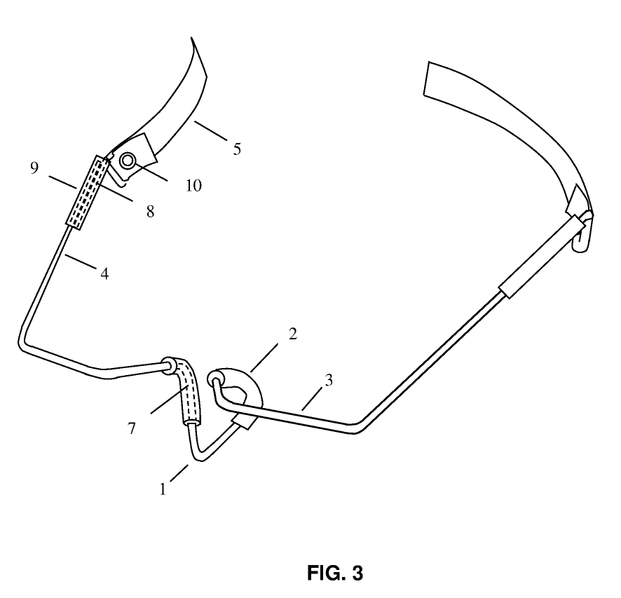

Referring now to FIG. 3, the detailed description of the device. Two curved nose pad segments 2 are covered with non-latex rubber sleeves 7 and joined together at the central point 1. The curved nose pad segments are extended side way to form a pair of frontal segments 3 and then angled backwards to form a pair of temple segments 4. Each of the two hook and loop fastening bands 5 is fixed by a ring 10 on a temple hook 8. Each of the two temple hooks is attached to a temple segment by inserting both temple segment and temple hook into a non-latex rubber temple sleeve 9.

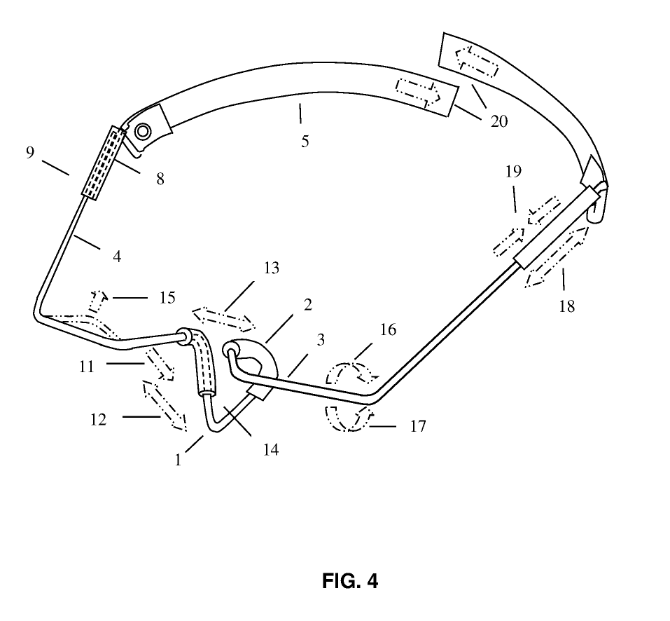

Referring now to FIG. 4, the adjustable components and adjustment methods of the device. The curved nose pad segments 2 can be bended towards 11 the central point 1 to increase their curvature to fit deeper facial contour or be bended away 12 from the central point to decrease their curvature to fit flatter facial contour. The distance 13 between two curved nose pad segments can be adjusted to fit either wider or narrower nose by bending the angle 14 at the central point. The frontal segments 3 can be bended upwards 15 to fit narrower face. The frontal segments 3 can be twisted clockwise 16 to fit deeper set eyes or counterclockwise 17 to fit shallower set eyes. After the nose pad and frontal frame adjustments, the central point should be adjusted to point away perpendicularly from the nose to allow room for the nose. The length of temple segment 4 can be extended by pulling the temple hook 8 inside the temple sleeve 9 outwards 18 to fit larger head size, or be reduced by pushing the temple hook inwards 19 to fit smaller head size. To apply pressure on the nasolacrimal ducts, wrap the hook and loop bands around the back of the head and then tighten them by reducing a desired amount 20 of length. Excessive tightness will not produce excessive pressure on the nasolacrimal ducts as the pressure is capped by the yield stress limit of the device frame. The required pressure varies from persons but can be determined by trial and error via leak checking using bitter taste test at home or dye disappearance test at clinic.

The invention claimed is:

1. A device for temporarily occluding the nasolacrimal ducts in adult patients to reduce outflow through the nasolacrimal ducts, the device comprising a steel wire device frame, steel wire hooks, rubber sleeves and hook and loop bands;
the steel wire device frame comprising:
a first curved nose pad segment and a second curved nose pad segment joined together at the central point, each of the first and second curved nose pad segments being formed to fit across the nasolacrimal duct at the nasal aspect of the orbital rim;
a first frontal segment and a second frontal segment, with the first and second curved nose pad segments being located between the first frontal segment and the second frontal segment;
a first temple segment and a second temple segment extended from first and second frontal segments;
the steel wire hooks comprising:
a first temple hook and a second temple hook;
the rubber sleeves comprising:
a first rubber sleeve covering the first curved nose pad segment and a second rubber sleeve covering the second curved nose pad segments;
a third rubber sleeve covering the first temple segment and the first temple hook and a fourth rubber sleeve covering the second temple segment and the second temple hook;
the hook and loop bands comprising:
a first hook and loop band attaching to the first temple hook and the second hook and loop band attaching to the second temple hook;

2. The device according to claim 1, wherein the device is flexible and can be bended by hand;
the hand adjustable items comprising:
the curvature of curved nose pad segments, which is adjustable by bending both curved nose pad segments towards the central point to increase their curvature to fit deeper facial contour or bending away from the central point to decrease their curvature to fit flatter facial contour;
the distance between two nose pad segments, which is adjustable by bending the angle at the central point to fit either wider or narrower nose;
the width of the frontal segments, which is adjustable by bending both frontal segments upwards to fit narrower face;
the angle between the curved nose pad segments and temple segments, which is adjustable by twisting both frontal segments clockwise to fit deeper set eyes or counterclockwise to fit shallower set eyes;
the position of the central point relative to the nose, which is adjusted to point away perpendicularly from the nose to allow room for the nose;
the length of temple segment, which is extendable by pulling the temple hook away from the temple sleeve to fit larger head size, or pushing the temple hook into the temple sleeve to fit smaller head size;
the pressure exerted on the nasolacrimal ducts, which can be controlled by adjusting the tightness of hook and loop bands around the back of the head; the proper pressure varies from persons but can be determined via trial and error using the bitter taste test or dye disappearance test.

3. The device according to claim 1, wherein the device is biocompatible;
the biocompatible items comprising:
the curved nose pad segments, which are covered with non-latex rubber sleeves;
the temple segments and temple hooks, which are covered with non-latex rubber sleeves.

4. The device according to claim 1, wherein the device is safe;
the embedded safety items comprising:
the device frame and temple hooks, which are coated with non-flammable plastic;
the hook and loop bands, which are made of flame retardant fabric;
the pressure applied on nasolacrimal ducts, which is capped by the yield stress limit of the device frame.

5. The flexible fitting method herein disclosed offers advantage over fixed fitting methods used by prior devices because it enables positive and complete occlusion.
